(19) United States
(12) Patent Application Publication
(10) Pub. No.: US 2016/0230139 A1
(43) Pub. Date: Aug. 11, 2016

(54) Self-Contained Device and System to Produce Ex-Vivo Autologous Whole Cell Tumor Vaccines

(71) Applicants: Xiaodong Wu, Miami, FL (US); Chandan Guha, Scarsdale, NY (US)

(72) Inventors: Xiaodong Wu, Miami, FL (US); Chandan Guha, Scarsdale, NY (US)

(21) Appl. No.: 14/948,377

(22) Filed: Nov. 22, 2015

Related U.S. Application Data
(60) Provisional application No. 62/083,169, filed on Nov. 22, 2014.

(51) Int. Cl. C12M 1/00; C12M 1/12; A61K 39/00

(52) U.S. Cl. CPC C12M 47/06; A61K 39/0011; C12M 25/02; C12M 23/34; C12M 47/10

(57) ABSTRACT

The invention disclosed herein aims to standardize and simplify the process of preparing Ex-Vivo autologous whole tumor cell vaccines. The present invention is a robust, stand-alone device and system for preparing autologous tumor cell vaccines in a completely self-contained sterile environment, and in a shortened time. This new device and system will process the extracted tumor with its associated stromal and endothelial cells into injectable tumor cell vaccines, administered automatically or semi-automatically. This invention incorporates a number of new biotechnologies to enhance therapeutic effects over other existing methods. This invention will allow a medical facility to prepare and administer autologous cancer cell vaccine therapy independently without having, or using, a GMP facility, while adhering to and maintaining GMP guidelines.

### CROSS-REFERENCE TO RELATED APPLICATIONS

This application claims the benefit of U.S. Provisional Application No. 62/083,169 filed on Nov. 22, 2014. The entire disclosure of this prior application is hereby incorporated by reference.

### STATEMENT REGARDING FEDERALLY SPONSORED RESEARCH OR DEVELOPMENT

This invention has been created without the sponsorship or funding of any federally sponsored research or development program.

### FIELD OF THE INVENTION

The present invention relates generally to a device and system for standardizing and simplifying the process of generating and preparing tumor vaccines as a form of immunotherapy.

### BACKGROUND

The outcomes of cancer treatment have been improved dramatically over the past few decades through the extensive efforts, in many disciplines, of cancer research and treatments. However, survival for middle-stage and late-stage cancer patients, of most tumor types, remains a serious challenge. Immunotherapy as a treatment modality for cancer has been under investigation for decades. Following the rapid advance of several important modern biotechnologies including genomics, epigenetics, and proteomics, cancer immunotherapy has gained significant new momentum in recent years.

Cancer vaccination is an active form of immunotherapy that stimulates the body's immune system to recognize and eradicate cancer cells. The basic principle is to activate antigen-presenting cells (APCs) to take up tumor-associated antigens (TAAs), present them to and activate the cytotoxic T cells (CTLs), which will in turn, target and kill the remaining tumor cells. The categories of cancer vaccine strategies include: 1) Whole Tumor Cell Vaccines; 2) Antigen-Based Vaccines; 3) APC-Based Vaccines; and Radiation-Mediated Vaccines.

Whole Tumor Cell Vaccines are derived from cancer cells that have been removed during surgery. The cells are treated (weakened or killed) in the lab, with radiation, freeze-thaw cycling, or other means, in order to terminate their ability to further divide. Varieties of adjuvant compounds can be added to enhance immune response. The cells are then injected into the patient intra-dermally (under the skin). The immune activation starts with the vaccine cells being taken up by antigen-presenting cells (APCs), such as dendritic cells and subsequently presented to CD4 and CD8 T-cells resulting in activation of cytotoxic T-cells (CTLs, CD8). This will lead to the recognition and killing of the remaining tumor cells by the matured CTLs.

Tumor cell vaccines are further divided into: a) autologous, meaning the vaccine is made from weakened or killed tumor cells taken from the same patient; b) allogeneic, meaning the cells for the vaccine come from someone other than the patient being treated; and c) gene-modified in which genes are inserted into the patient's tumor cells causing immunostimulatory proteins, such as GM-CSF, IL-2 to be expressed on their surface. Allogeneic vaccines are easier to make than autologous vaccines, but it is not yet clear if it can be as effective as the autologous approach.

Antigen-Based Vaccines stimulate the immune system by injecting one or more purified tumor-specific antigens, rather than whole tumor cells that contain many thousands of antigens. Antigen-based vaccines may be furthered divided into four groups: 1) Peptide-based vaccines, which use tumor-specific protein fragments with modified segments as immune activating antigens; 2) Heat shock protein (HSP) vaccines, in which HSP-tumor peptides complex functions as the tumor-specific antigen, activating the immune response through dendritic cell's HSP receptors; 3) DNA vaccines, in which DNA-containing genes of tumor-specific protein is injected into patient to produce tumor-specific protein as immune stimulator; and 4) Viral and bacterial vector vaccine, an alternate way of DNA vaccination by using bacteria or viruses as carriers of the DNA. Although antigen-based vaccines are tumor specific, they are not patient specific, and therefore have inherent inconsistency in clinical response between patients.

The current focus of APC-based vaccines is the use of dendritic cells (DC) which are the most potent APCs and are up to 1000 times more effective than other types of APCs in stimulating antigen specific T cells. DC-based vaccines use patient-specific dendritic cells, isolated/multiplied from the patient's peripheral blood and pulsed with dead tumor cells/fragments or tumor-associated antigens in the lab. The matured dendritic cells are then injected back into the patient, where they should provoke an immune response to cancer cells in the body. Sipuleucel-T (Provenge), an example of a dendritic cell vaccine, is the first FDA approved cancer vaccine for treating advanced prostate cancer.

Radiation-Mediated Vaccines involves the use of ionizing radiation, which when used for radiation therapy, causes tumor cell death. Recent research has demonstrated the efficacy of using radiation therapy as a novel strategy of in situ whole tumor cells vaccination. The idea is to harness radiation induced tumor dead cells as a potential source of tumor-associated antigens for immunotherapy. Early studies have shown that a combination of radiation therapy with immunotherapies, including: a) antibody blockade of negative T cell checkpoints such as CTLA-4 and PD-1; b) antibody agonist of co-stimulatory receptors such as CD137; and c) Expansion of APCs by administrating Flt3L, have resulted in markedly improved treatment outcomes.

In addition to inducing tumor cell death as a source of tumor antigens, many studies reveal that radiation itself can increase antigen presentation by generating intracellular tumor peptides and up-regulating the expression of Class I and II major histocompatibility complex (MHC) molecules resulting in enhancement of tumor immunogenicity. Radiation can also induce the release of some immunogenic cytokines and chemokines, such as IFNs that induce DC maturation and CXCL16 that attracts CTLs to the tumor site.

Apart from radiation-mediated in situ vaccination strategy, preparation for other cancer vaccines are generally labor intensive, time consuming and costly. Of the first three categories outlined above, whole tumor cell vaccine is logically simpler and in some ways more cost effective. The advantages of using autologous tumor cells are that they are a good source of TAAs and are patient-specific. They can be administered directly without ex-vivo preparation of dendritic cells.

Autologous tumor cell vaccination was pioneered by M. G Hanna. Following his pre-clinical animal works in 1970s, Hoover et al. conducted a clinical trial for patients with stage II/III colorectal cancer using irradiated autologous tumor cells mixed with BCG, randomized versus surgery alone. Subgroup analysis revealed significant overall and disease-free survival for vaccinated patients. In addition, delayed type hypersensitivity (DTH) reactions to autologous tumor cells suggested the presence of tumor-specific immunity. The promising results prompted and initiated additional clinical trials for colorectal cancer and other tumors. The investigations of Hanna and his colleagues were further developed into a patented product of a colorectal cancer vaccine service, OncoVAX®, currently owned by Vaccinogen.

A typical OncoVAX® service starts with Vaccinogen taking the tumor sample from each patient to its good manufacturing practices (GMP) facility. The technicians sterilize the tumor, individual cells are then selected, extracted and irradiated with a high dose of radiation. The vaccine consisting of tumor cell lysates mixed with TICE BCG are then prepared into injection vials. The vaccines are injected into the patient's skin in four doses over the first six months after the surgery.

The injections produce a delayed-type hypersensitivity (DTH) response, which indicates that the body's own T-cells will respond to tumor antigens. A randomized, 254-patient Phase IIIa clinical trial for OncoVAX® at twelve different hospitals in The Netherlands has been completed. The results, published in The Lancet and in Vaccine, demonstrated a statistically significant increased 5-year overall survival rate and increased recurrence-free survival by log-rank analysis, as well as a dramatic increase in the time to tumor progression rate in treated patients. At a median follow-up period of 5.8 years for Stage II colon cancer patients, the Kaplan-Meier 5-year recurrence-free survival rate was increased by 41% with a 21.3% death/recurrence rate for the patients treated with OncoVAX®, compared to 37.7% for the surgery-alone control group (p-value of 0.008). Treated patients also demonstrated a statistically significant 33% increase in 5-year overall survival (p-value of 0.014) and an 80% reduction in tumor progression rate at 18 months following treatment with OncoVAX®. A pivotal Phase tub trial is currently on going under a Special Protocol Assessment (SPA) and Fast Track Designation. Vaccinogen anticipates to receive FDA approval for OncoVAX® in 2015.

### SUMMARY OF THE INVENTION

With OncoVAX® as an example, all current whole tumor cell vaccinations require a GMP facility and a long processing time (several weeks). The invention disclosed herein aims at standardizing and simplifying the process of preparing autologous tumor cell vaccines with significantly shortened processing time, by developing a robust, stand-alone device and system, currently registered as AutoVAX®, in a completely self-contained sterile environment. This new device and system will process the extracted tumor with its associated stromal and endothelial cells into injectable tumor cell vaccines, administered automatically or semi-automatically. This invention incorporates a number of new biotechnologies to enhance therapeutic effects over other existing methods. This invention will allow a medical facility to prepare and administer autologous cancer cell vaccine therapy independently without having, or using, a GMP facility, while adhering to and maintaining GMP guidelines.

### BRIEF DESCRIPTION OF THE DRAWINGS

FIG. 1 is a flow chart showing the functional schematics of the system of the invention as described herein.

FIG. 2 is a diagram of the components of the device of the invention as described herein.

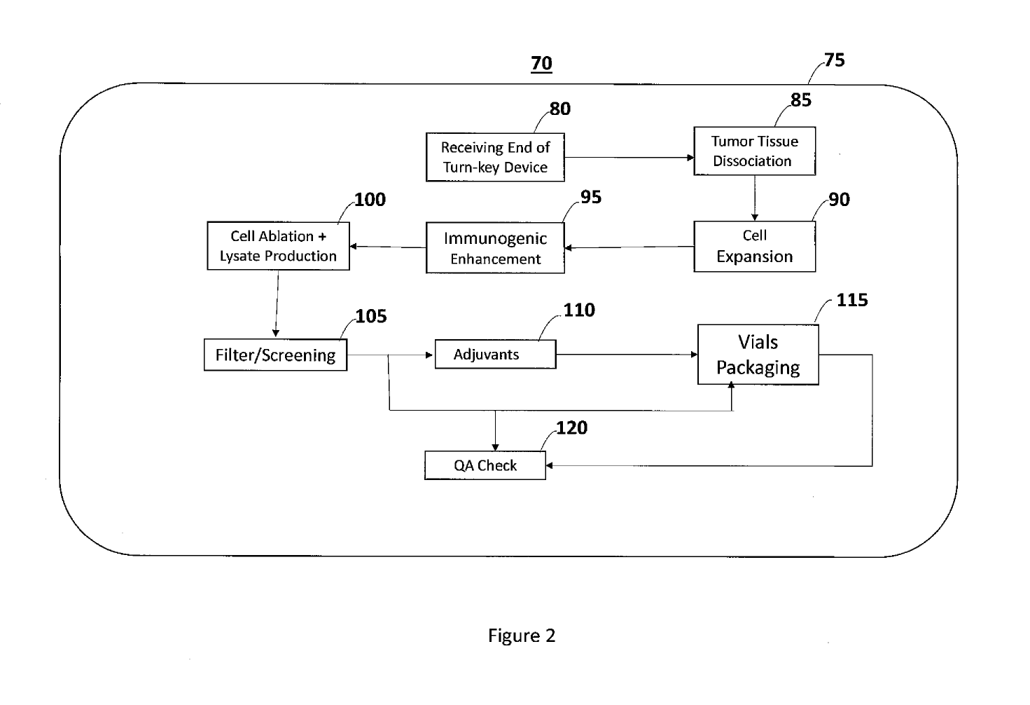

### DESCRIPTION OF THE INVENTION

Before the subject invention is described further, it is to be understood that the invention is not limited to the particular embodiments of the invention described below, as variations of the particular embodiments may be made and still fall within the scope of the invention. It is also to be understood that the terminology employed is for the purpose of describing particular embodiments, and is not intended to be limiting.

The details of one or more embodiments of the invention are set forth in the accompanying drawings and the description below. Other features, objects, and advantages of the invention will be apparent from the description and drawings, and from the claims. In the following description, numerous specific details are set forth to provide a thorough understanding of the embodiments. One skilled in the art to which this invention belongs will recognize, however, that the techniques described can be practiced without one or more of the specific details, or with other methods, components, materials, etc. In other instances, well known structures, materials or operations are not shown or described in detail to avoid obscuring certain aspects.

In this specification, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

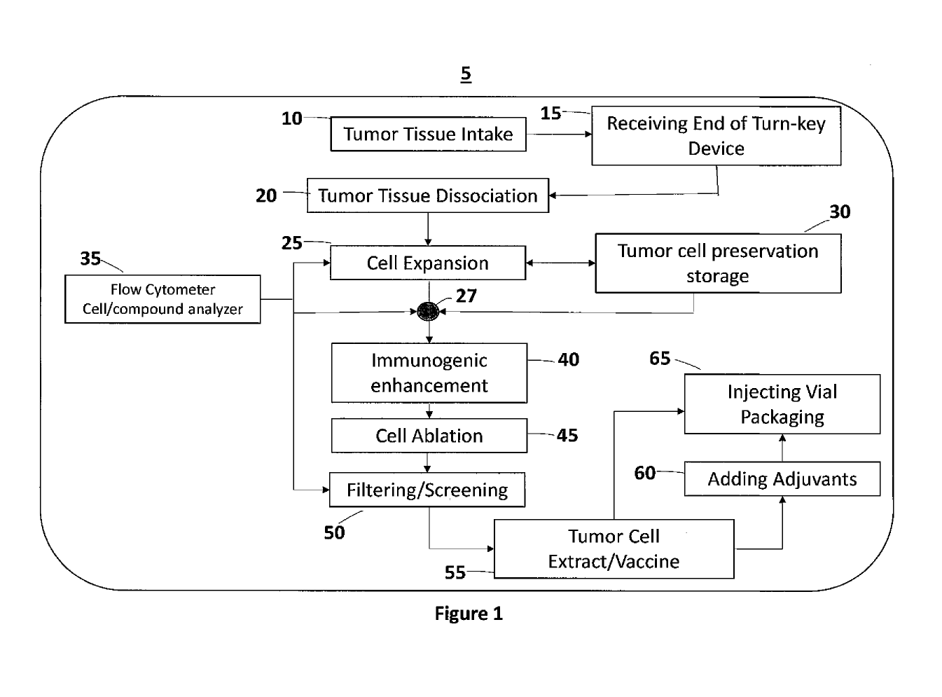

The invention disclosed herein is designed to be a clinical-grade, turn-key device and system 5 for producing autologous cancer cell vaccines. As shown in FIG. 1, tumor tissue, or part of the tumor, will be first be removed from the patient under sterile conditions as part of the tumor tissue intake process 10. The removed tumor fragment will be transferred using a sterile instrument, to the receiving end of the device 15, where the enzymatic dissociation of tumor/stromal/endothelial cell extraction 20 will take place. The extracted tumor/stromal/endothelial cells will undergo expansion 25, immunogenic enhancement 40, and cell ablation 45. The extracted tumor cells undergo a screening and filtering process 50 to eliminate immune suppression factors. The tumor cell vaccine 55 is finally prepared into vials 65 with, or without, adjuvants 60 immunotherapy agents. The entire process is carried out in a self-contained sterile environment 5. The system has a built in quality assurance feature comprising a flow cytometer cell/compound analyzer 35, which runs throughout the processing of the tumor cell vaccine, including prior to packaging.

The device 70 for effectuating the systematic processing of autologous cancer cell vaccines includes the following key components: A self-contained sterile housing 75; a receiving end 80 for accepting and storing tumor tissue with its associated stromal and endothelial cells; adjacent to the receiving end, a compartment for tumor fragment dissociation and vaccine cells extraction 85, a compartment for vaccine cells expansion 90; a compartment for immunogenic enhancement 95; a compartment for cell ablation and tumor cell lysates production 100; a compartment for the screening and filtering of the secreted immune suppressive factors 105; a compartment for integration of immunotherapy adjuvants 110, such as Flt3L and GM-CSF; a compartment for vaccine vials packaging 115; and within the system, a Quality Assurance (QA) subunit 120 that performs a quality check and cell analysis. FIG. 1 is a functional schematic of the device as described herein.

In making a robust, stand-alone turn-key system, the invention incorporates the following unique proprietary features:

Tumor Tissue Intake with Minimally Invasive Procedure.

Traditionally, tumor or its fragments are obtained from surgical resection, where the invention uses minimally invasive procedures. More specifically, the invention uses tumor tissues obtained through minimally invasive interventional radiological procedures, such as image-guided needle biopsy, and then process the tumor tissue. The minimally invasive tumor extraction is used for patients who have multiple metastatic tumors and/or are contra-indicated for open surgery. Minimally invasive procedures have the advantage over open surgery by preserving the strength of the patient's immune system.

Vaccination Cells.

Unlike traditional whole tumor cell vaccination, the disclosed invention includes not only tumor cells, but also tumor stromal cells and endothelial cells as the source of vaccination. Recent research has revealed that tumor stromal cells and tumor endothelial cells provide a pro-tumor growth environment and should also be targeted, therefore including stromal and endothelial lysates as a source of antigens in vaccination is synergetic to tumor cell vaccination and is expected to enhance therapeutic effects. This strategy also simplifies the cell extraction process, as one no longer needs to isolate tumor cells.

Vaccine Cell Expansion.

This invention proposes to use Induced Pluripotent Stem Cells (IPS), to expand the vaccine cell counts (tumor/stromal/endothelia). This presents several unique advantages, including but not limited to:
The quantity of vaccine cell lysates needs to meet a certain threshold to warrant a positive clinical response. In case the size/quantity of the dissected tumor tissue is insufficient, the IPS cell expansion provides an effective solution.
Multiple vaccination treatments are likely necessary to maintain long-term disease control. As long as the tumor genotype remains unchanged, IPS cell expansion methods provides long-term treatment without additional invasive procedures of obtaining tumor tissue for vaccine preparation. This is by expanding and storing sufficiently large quantities of vaccine cells from the initial dissected tumor tissues.

Immunogenicity Enhancement and Tumor Cell Ablation.

One of the challenges in whole tumor cell vaccination is associated with the fact that live tumor cells could be poorly immunogenic, and are shown to secrete soluble factors, such as: vascular endothelial growth factor to suppress DCs differentiation and maturation; soluble Fas ligand to induce lymphocyte apoptosis; or soluble MICA products to inhibit NKG2D mediated killing by immune cells. In addition, IL-10 and TGF-β released by tumor cells could inhibit DC and T cell functions. Galectin-1 and indoleamine 2,3-dioxygenase also inhibit T cell activation. Therefore tumor cell ablation for vaccine preparation should be combined with a means of enhancing or stimulating tumor cell immunogenicity.

Commonly used death-initiating stimuli include repetitive freeze-thaw cycles, exposure to ultraviolet (UV) ray, HOCL oxidation, exposure to x-rays or gamma rays and viral infection. Currently, single ablation method is generally used in the preparation of whole tumor cell vaccine. With a single method, it is very difficult to achieve cell killing and at the same time to enhance the tumor cell immunogenicity.

This invention proposes to use a multi-step process with optimized sequence to enhance tumor-specific immunogenicity, namely to induce tumor-specific antigens first, and then to ablate the tumor cells.

At least three methods will be available to induce tumor-specific antigens: 1) Using low dose ionizing radiation (<50 cGy, for example) to generate intracellular peptides and increase MHC-peptides expression; 2) Using heat treatment with low intensity ultrasound or microwave at 41-45° C. to induce HSP-tumor peptide complex (based on the recent research finding in Dr. Guha's Lab); and 3) Using combined low dose radiation and low intensity ultrasound or microwave to induce tumor antigens and their expression.

The system will provide sufficient flexibility to allow for other methods of antigen induction, such as light-heavy ion irradiation. This step can be very advantageous in eliminating common antigens expressed on both the tumor and normal cells, which is a common challenge in current whole tumor cell vaccination strategies.

Following antigen induction, multiple options will be available for cell ablation including freeze-thaw cycles, UV exposure, IR (ionizing radiation including heavy ion beams) exposure, HOCL oxidation and high intensity thermal treatment with ultrasound or microwaves. Cell ablation can be done with built-in, external sources or combined.

If an antigen induction process is used in the vaccine preparation, the same stimulus (low dose radiation, low intensity heat treatment, or combined) might be applied for antigen induction in situ locally, or systemically, to microscopic disease, in concert with the vaccine administration.

Screening Secreted Factors.

This invention proposes to include a screening and filtering/washing process to eliminate factors secreted by tumor cells that may inhibit therapeutic immune response and preserve factors that may boost immune efficacy, such as IFNs and CXCL16.

Adjuvant Compounds.

In order to enhance uptakes of cell lysates following the injection, this invention proposes to mix the tumor cell lysates most effective compounds as adjuvants, such as Flt3L (by Celldex) and GM-CSF (Leukine by Amgen). In conjunction with the administration of the tumor cell vaccine generated by this invention, concomitant infusions of negative immune checkpoint blockades such as PD1 antibody and CTLA-4 antibody are also proposed.

As various changes may be made in the above-described subject matter without departing from the scope and the spirit of the invention, it is intended that all subject matter contained in the above description, or shown in the accompanying drawings, will be interpreted as descriptive and illustrative, and not in a limiting sense.

### EQUIVALENTS

Those skilled in the art will recognize, or be able to ascertain using no more than routine experimentation, many equivalents to the specific embodiments of the invention described herein. Such equivalents are intended to be encompassed by the claims.

What is claimed is:

1. A stand-alone turn-key device for producing and processing autologous tumor cell vaccines in a self-contained sterile environment, comprising:
a. A self-contained and sterile housing, having;
i. A receiving end for accepting and storing tumor tissue with associated stromal and endothelial cells;
ii. A compartment adjacent to the receiving end for processing the enzymatic dissociation and extraction of the tumor's stromal and endothelial cells;
iii. A compartment for accomplishing cell expansion, employing Induced Pluripotent Stem Cells (IPS);
iv. A compartment for the processing of immunogenic enhancement of the tumor cells;
v. A compartment for conducting tumor cell ablation and tumor cell lysate production;
vi. A compartment for screening and filtering of secreted immune suppressive factors generated from the tumor cell ablation process, and then isolating the filtered extract into an injectable form for packaging;
vii. A compartment for the integration of immunotherapy adjuvants to the filtered extract prior to packaging;
viii. A compartment for packaging the filtered extract as tumor cell vaccines in a vial for injection into a patient; and
ix. A quality assurance subunit for performing a quality check and cell analysis on the packaged vaccine prior to use on the same patient.

2. A turn-key system for producing autologous tumor cell vaccines in a self-contained sterile environment, comprising:
a. Identifying a patient with a tumor;
b. Non-surgically, removing the tumor with associated stromal and endothelial cells from the patient, under sterile conditions;
c. Transferring the removed tumor with associated stromal and endothelial cells, using a sterile instrument, to a turn-key device comprising;
i. A self-contained and sterile housing, having;
1. A receiving end for accepting and storing tumor tissue with associated stromal and endothelial cells;
2. A compartment adjacent to the receiving end for processing enzymatic dissociation and extraction of the tumor's stromal and endothelial cells;
3. A compartment for accomplishing cell expansion, employing Induced Pluripotent Stem Cells (IPS);
4. A compartment for the processing of immunogenic enhancement of the tumor cells;
5. A compartment for conducting tumor cell ablation and tumor cell lysate production;
6. A compartment for screening and filtering of secreted immune suppressive factors generated from the tumor cell ablation and cell lysate production, and then isolating the filtered extract into an injectable form for packaging;
7. A compartment for the integration of immunotherapy adjuvants into the filtered extract prior to packaging;
8. A compartment for packaging the filtered extract as a tumor cell vaccine in a vial for injection into the same patient; and
9. A quality assurance subunit for performing a quality check and cell analysis on the packaged vaccine prior to use on the same patient.
d. Using the turn-key device to complete enzymatic dissociation of the tumor with associated stromal and endothelial cells;
e. Using the turn-key device to extract the tumor's stromal and endothelial cells;
f. Using the turn-key device to accomplish cell expansion of the tumor's stromal and endothelial cells, whereby Induced Pluripotent Stem Cells (IPS) are employed to increase the cell count;
g. Using the turn-key device to accomplish immunogenic enhancement of the extracted cells, employing low dose ionizing radiation to generate intracellular peptides and increase MHC-peptides expression;
h. Using the turn-key device to accomplish cell ablation of the extracted cells;
i. Generating the tumor cell vaccine consisting of the components of the extracted tumor cells;
j. Using the turn-key device to prepare and package the tumor cell vaccine into vials, optionally adding adjuvant immunotherapy agents prior to packaging.

3. The system as in claim 2, whereby immunogenic enhancement of the extracted cells is accomplished by using heat treatment with low intensity ultrasound at 41-45° C. to induce a Heat Shock Protein (HSP)-tumor peptide complex.

4. The system as in claim 2, whereby immunogenic enhancement of the extracted cells is accomplished by using heat treatment with low intensity microwave at 41-45° C. to induce a Heat Shock Protein (HSP)-tumor peptide complex.

5. The system in claim 2, whereby immunogenic enhancement of the extracted cells is accomplished by using combined low dose radiation and low intensity ultrasound at 41-45° C. to induce tumor antigens and their expression.

6. The system in claim 2, whereby immunogenic enhancement of the extracted cells is accomplished by using combined low dose radiation and low intensity microwave at 41-45° C. to induce tumor antigens and their expression.

7. The system as in claim 2 whereby cell ablation is accomplished by subjecting the extracted cells to freeze-thaw cycles.

8. The system as in claim 2 whereby cell ablation is accomplished by subjecting the extracted cells to UV exposure.

9. The system as in claim 2 whereby cell ablation is accomplished by subjecting the extracted cells to Ionizing Radiation (IR) exposure, including heavy ion beams.

10. The system as in claim 2 whereby cell ablation is accomplished by subjecting the extracted cells to HOCL oxidation and high intensity thermal treatment with ultrasound.

11. The system as in claim 2 whereby cell ablation is accomplished by subjecting the extracted cells HOCL oxidation and high intensity thermal treatment with microwaves.
